(19) United States
(12) Patent Application Publication
(10) Pub. No.: US 2023/0159334 A1
(43) Pub. Date: May 25, 2023

(54) NITROGEN-CONTAINING CARBON MATERIAL AND METHOD FOR PRODUCING SAME

(71) Applicants: NICHIA CORPORATION, Anan-shi (JP); NATIONAL UNIVERSITY CORPORATION CHIBA UNIVERSITY, Chiba (JP)

(72) Inventors: Yasuhiro Yamada, Chiba (JP); Ryouhei Kawai, Chiba (JP); Ryoushin Suzuki, Chiba (JP); Hiroomi Taguchi, Yokohama-shi (JP)

(73) Assignees: NICHIA CORPORATION, Anan-shi (JP); NATIONAL UNIVERSITY CORPORATION CHIBA UNIVERSITY, Chiba (JP)

(21) Appl. No.: 18/058,690

(22) Filed: Nov. 23, 2022

(30) Foreign Application Priority Data
Nov. 24, 2021 (JP) 2021-190446

(51) Int. Cl. C01B 32/05; B01J 21/18; B01J 37/08; B01J 20/20; B01J 20/30; C07D 471/04; H01M 10/052; H01M 4/60; H01M 4/90

(52) U.S. Cl. CPC C01B 32/05; B01J 21/18; B01J 37/084; B01J 20/20; B01J 20/3078; C07D 471/04; H01M 10/052; H01M 4/608; H01M 4/9008; C01P 2002/52; C01P 2002/85; C01P 2002/82; C01P 2006/40

(57) ABSTRACT

A nitrogen-containing carbon material includes carbon atoms, nitrogen atoms, and halogen atoms. The nitrogen-containing carbon material has a ratio of a number of moles of pyridinic nitrogen atoms to a total number of moles of the nitrogen atoms that is higher than 59% and a total content ratio of the nitrogen atoms with respect to the nitrogen-containing carbon material that is 7 at % or higher. The nitrogen-containing carbon material includes a fused polycyclic aromatic moiety formed by condensation of three or more aromatic rings, and the fused polycyclic aromatic moiety includes a partial structure for two pyridinic nitrogen atoms to be linked to each other through two carbon atoms.

### CROSS-REFERENCE TO RELATED APPLICATIONS

This application claims priority to Japanese Patent Application No. 2021-190446, filed on Nov. 24, 2021, the disclosure of which is hereby incorporated by reference in its entirety.

### BACKGROUND

### Field of the Invention

The present disclosure relates to a nitrogen-containing carbon material and a method for producing the nitrogen-containing carbon material.

### Description of the Related Art

Carbon materials are materials for which application thereof are expected in various fields. Especially, for nitrogen-containing carbon materials into which pyridinic nitrogen atoms are introduced, various applications are expected such as, for example, an oxygen reducing catalysts, and electrode materials for lithium sulfur batteries, and many examples of synthesis of carbon materials including pyridinic nitrogen atoms have been reported. Masatoshi Murata, Yasuhiro Yamada, Shingo Kubo, and Satoshi Sato, Structural Control of a Nitrogen-Containing Carbon Material Applying Computational Chemistry, (Poster Presentation No. P-09, Joint Symposium for Graphene and Graphene Oxide, Akihabara Convention Hall, Tokyo, Dec. 8, 2017) discloses an example where 4,7-dichloro-1,10-phenanthroline into which halogen atoms were introduced was baked at 873K aiming at controlling the structure of 1,10-phenanthroline structure in a carbon material. It was reported that the ratio of the pyridinic nitrogen atoms in the nitrogen in the carbon material obtained according to this method was 59%.

### SUMMARY

According to a first exemplary embodiment can be provided a nitrogen-containing carbon material including carbon atoms, nitrogen atoms, and halogen atoms. The nitrogen-containing carbon material may have a ratio of a number of moles of pyridinic nitrogen atoms to a total number of moles of the nitrogen atoms that is higher than 59% and may have a total content ratio of the nitrogen atoms with respect to the nitrogen-containing carbon material that is 7 at % or higher. The nitrogen-containing carbon material may include a fused polycyclic aromatic moiety that is formed by condensation of three or more aromatic rings, and the fused polycyclic aromatic moiety may include a partial structure for two pyridinic nitrogen atoms to be linked to each other through two carbon atoms.

According to a second exemplary embodiment can be provided a method for producing the nitrogen-containing carbon material. The method may include providing a nitrogen-containing aromatic compound that includes a phenanthroline structure and that includes a halogen atom as a substituent thereof; and carbonizing the nitrogen-containing aromatic compound by applying a heat treatment at 200° C. or higher and lower than 600° C.

### BRIEF DESCRIPTION OF THE DRAWINGS

FIG. 1 is an exemplary X-ray photoelectron spectroscopy (XPS) spectrum of a is orbital of nitrogen in a nitrogen-containing carbon material according to Example 2.

FIG. 2 is an exemplary XPS spectrum of a is orbital of nitrogen in a nitrogen-containing carbon material according to Example 3.

FIG. 3 is an exemplary XPS spectrum of a is orbital of nitrogen in a nitrogen-containing carbon material according to Example 5.

FIG. 4 is an exemplary Raman spectroscopic spectrum of a nitrogen-containing carbon material according to Example 1.

FIG. 5 is an exemplary Raman spectroscopic spectrum of a nitrogen-containing carbon material according to Example 3.

### DETAILED DESCRIPTION

The term “step” as used herein encompasses not only an independent step but also a step not clearly distinguishable from another step as long as the intended purpose of the step is achieved. If multiple substances correspond to a component in a composition, the content of the component in the composition means the total amount of the multiple substances present in the composition unless otherwise specified. Further, upper limit and lower limit values that are described for a numerical range in the present specification can be arbitrarily selected and combined. Embodiments of the present disclosure will now be described in detail. The embodiments described below are exemplifications of a nitrogen-containing carbon material and a method for producing the same for embodying the technical ideas of the present disclosure, and the present disclosure is not limited to the nitrogen-containing carbon material and the method for producing the same described below.

### Nitrogen-Containing Carbon Material

A nitrogen-containing carbon material includes carbon atoms, nitrogen atoms, and halogen atoms. As to the nitrogen-containing carbon material, the ratio of the number of moles of pyridinic nitrogen atoms to the total number of moles of the nitrogen atoms may be higher than 59% and the total content ratio of the nitrogen atoms with respect to the nitrogen-containing carbon material may be 7at % or higher. The nitrogen-containing carbon material includes a fused polycyclic aromatic moiety that is formed by condensation of three or more aromatic rings and, the fused polycyclic aromatic moiety includes, a partial structure for the two pyridinic nitrogen atoms to be linked to each other through two carbon atoms.

A nitrogen-containing carbon material produced according to a producing method described later may include the pyridinic nitrogen atoms at a high content rate. In the case where the nitrogen-containing carbon material including the pyridinic nitrogen atoms at a high content ratio is applied to, for example, an electrode of a lithium sulfur battery, adsorption of a lithium polysulfide may be promoted and the shuttle effect of the lithium polysulfide may be suppressed. The cycle property of the lithium sulfur battery may thereby be improved. The nitrogen-containing carbon material including the pyridinic nitrogen atoms at a high content ratio may be excellent in the adsorption capacity for carbon dioxide. The nitrogen-containing carbon material may thereby be applied as, for example, a carbon dioxide sensor. The nitrogen-containing carbon material including the pyridinic nitrogen atoms at a high content ratio may have an electrode catalytic activity for a redox reaction, and this nitrogen-containing carbon material may therefore be applied as an electrode of a fuel battery. In addition, the nitrogen-containing carbon material including the pyridinic nitrogen atoms at a high content ratio may be advantageous for uses such as a catalyst carrier.

It may be considered that, in the nitrogen-containing carbon material, a portion of the carbon atoms constituting the carbon material is substituted by nitrogen atoms. In other words, it may be considered that the nitrogen-containing carbon material has a structure in which nitrogen atoms are doped into the carbon material. The carbon material may be constituted mainly by an sp²-type carbon. The “carbon material” as used herein means a carbon material whose content of carbon is 50at % or higher and preferably 55 at % or higher from the composition ratios determined by an elemental analysis and that has a G-band (a range that is 1,570 cm⁻¹ or greater and 1,600 cm⁻¹ or smaller) observed by Raman spectroscopy analysis. For the carbon material, a D-band (a range that is 1,300cm⁻¹ or greater and 1,400 cm⁻¹ or smaller) may be also observed by inclusion therein of a defect structure of the nitrogen atoms and the like. It is generally stated that the G-band is related to a graphene structure or a chemical structure similar to the graphene structure. The D-band reflects the presence of a structural defect or a function group included in the graphene structure or the chemical structure similar to the graphene structure, and both of the G-band and the D-band may be observed for the nitrogen-containing carbon material. In addition, for the nitrogen-containing carbon material, such bands may be observed as a D′-band (a range that is 1,600 cm⁻¹ or greater and 1,650 cm⁻¹ or smaller), a 2D-band (a range that is 2,650 cm⁻¹ or greater and 2,750 cm⁻¹ or smaller), a D+G-band (a range that is 2,800 cm⁻¹ or greater and 3,000 cm⁻¹ or smaller), and a 2G-band (a range that is 3,100 cm⁻¹ or greater and 3,300 cm⁻¹ or smaller). The position of each of these bands herein is observed when the wavelength of the excitation light source is 532 nm.

The nitrogen atoms substituting a portion of the carbon atoms constituting the carbon material portion of the nitrogen-containing carbon material may include electrically neutral basal nitrogen present in a basal surface and including sp²-type carbon atoms linked to each other through a double bond, and edge nitrogen present in edge portions, and may also include quaternary nitrogen atoms (Q-N) having a positive charge. The basal nitrogen may take a form represented by any one of structural formulae (a) to (d) below from the bonding mode thereof. The edge nitrogen may also take a form represented by any one of structural formulae (e) to (i) below from the bonding mode thereof. The portion depicted by a dotted line in each of the structural formulae below indicates a resonance structure through the sp²-type carbon atoms and nitrogen atoms. The basal nitrogen is a tertiary nitrogen atom bonded with three sp²-type carbon atoms, and the edge nitrogen is a primary or a secondary nitrogen atom bonded with one or two sp²-type carbon atom(s). These may be distinguished from each other using X-ray photoemission spectroscopy (XPS) spectrum measurement for a is orbital of nitrogen.

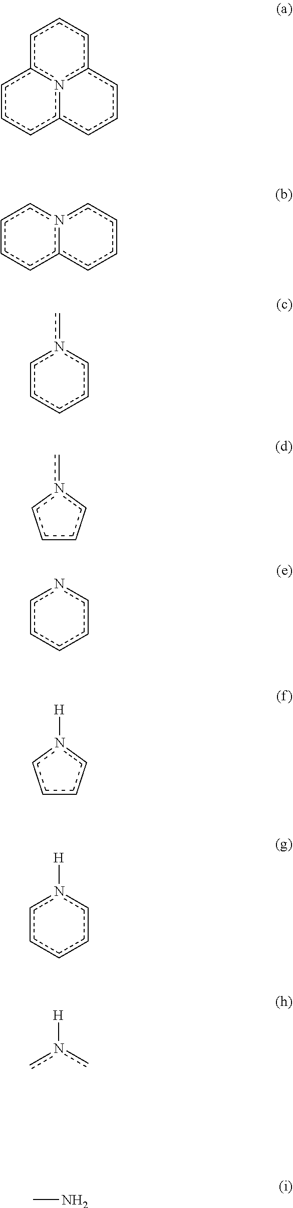

The structural formula (a) represents a nitrogen-containing structure in which one tertiary nitrogen atom is positioned inside three six-membered ring structures, and is referred to as “T3”. The structural formula (b) represents a nitrogen-containing structure in which one tertiary nitrogen atom is positioned inside two six-membered structure rings, and is referred to as “T2”. The structural formula (c) represents a nitrogen-containing structure in which one tertiary nitrogen atom is positioned inside one six-membered ring structure, and is referred to as “T1”. The structural formula (d) represents a nitrogen-containing structure in which one tertiary nitrogen atom is positioned inside one five-membered ring structure, and is referred to as “T1P”.

The structural formula (e) represents a nitrogen-containing structure in which one secondary nitrogen atom is positioned inside one six-membered ring structure, has a structure similar to a benzene ring, and is referred to as “pyridinic nitrogen”. The structural formula (f) represents a nitrogen-containing structure in which one secondary nitrogen atom is positioned inside one five-membered ring structure, and is referred to as “pyrrolic nitrogen”. The structural formula (g) represents a nitrogen-containing structure in which one secondary nitrogen atom is positioned inside one six-membered ring structure, and is referred to as “S1”. The structural formula (h) represents a nitrogen-containing structure that includes a secondary nitrogen atom having no ring structure, and is referred to as “S0”. The structural formula (i) represents a nitrogen-containing structure that includes a primary nitrogen atom, and is referred to as “NH₂”. S0 and NH₂ are collectively referred to also as “amine-type nitrogen”. For the above classification of nitrogen atoms using the basal nitrogen and the edge nitrogen, Non-Patent Literature, Y. Yamada, H. Tanaka, S. Kubo, and S. Sato, “Unveiling Bonding States and Roles of Edges in Nitrogen-Doped Graphene Nanoribbon by X-ray Photoelectron Spectroscopy”, Carbon 185 (2021), 342-367 was referred to.

The total content ratio of the nitrogen atoms included in the nitrogen-containing carbon material may be, for example, 7 at % or higher. The total content ratio of the nitrogen atoms may be preferably 8 at % or higher, 9 at % or higher, or 10 at % or higher. The total content ratio of the nitrogen atoms may be, for example, 25 at % or lower and may be preferably 20 at % or lower. Degradation of the electric conductivity of the carbon material may thereby be suppressed. The total content ratio of the nitrogen atoms in the nitrogen-containing carbon material may be calculated from elemental analysis values of the nitrogen-containing carbon material. For example, the composition ratios of the nitrogen-containing carbon material may be calculated by dividing the elemental analysis value of each of the constituent elements by the atomic weight thereof, and the total content (at %) of the nitrogen atoms may be calculated based on the obtained composition ratios.

As to the nitrogen-containing carbon material, the ratio of the number of moles of the pyridinic nitrogen atoms to the total number of moles of the nitrogen atoms may be higher than, for example, 59%. The ratio of the number of moles of the pyridinic nitrogen atoms may be preferably 65% or higher, 75% or higher, or 85% or higher. The ratio of the number of moles of the pyridinic nitrogen atoms to the total number of moles of the nitrogen atoms may be, for example, 99% or lower, or 95% or lower. It may be stated that the nitrogen-containing carbon material more selectively includes the pyridinic nitrogen atoms as the ratio of the number of moles of the pyridinic nitrogen atoms to the total number of moles of the nitrogen atoms is closer to 100%. It may be stated that the nitrogen-containing carbon material is excellent in the controllability of the structure. The ratio of the number of moles of the pyridinic nitrogen atoms to the total number of moles of the nitrogen atoms may be analyzed by measuring, for example, an XPS spectrum of the is orbital of nitrogen using XPS.

The nitrogen-containing carbon material may include a halogen atom. Examples of the halogen atom include fluorine atom, chlorine atom, bromine atom, iodine atom, and the like, and the nitrogen-containing carbon material may include at least one selected from the group consisting of the above. The nitrogen-containing carbon material may include at least one selected from the group consisting of, preferably, chlorine atom, bromine atom, and iodine atom, as the halogen atom. The halogen atom may be included in the nitrogen-containing carbon material, being derived from the producing method described later. It may be considered that the halogen atom is each covalently bonded with a portion of the carbon atom that constitute a fused polycyclic aromatic moiety formed by condensation of, for example, three or more aromatic rings.

The content ratio of the halogen atom included in the nitrogen-containing carbon material may be, for example, 0.01 at % or higher and 30 at % or lower. The content ratio of the halogen atom may be 0.1 at % or higher, 0.3 at % or higher, 0.5 at % or higher, or 1 at % or higher, and may be preferably 5 at % or lower, 3 at % or lower, 2 at % or lower, or 1.5 at % or lower. The content ratio of the halogen atom in the nitrogen-containing carbon material is determined from the difference between the sum of the rates of carbon, hydrogen, and nitrogen that are experimentally obtained by the elemental analysis, and the total (100 wt %) of the elemental analysis value that is theoretically determined.

As to the nitrogen-containing carbon material, the mole ratio of the content of the nitrogen atoms to the content of the halogen atoms (that is, the number of moles of the nitrogen atoms/the number of moles of the halogen atoms) may be, for example, 0.3 or greater and 2,500 or smaller. The mole ratio of the content of the nitrogen atoms to the content of the halogen atoms may be preferably 1 or greater, 2 or greater, or 6 or greater, and may be preferably 2,000 or smaller, 1,500 or smaller, or 1,000 or smaller.

As to the nitrogen-containing carbon material, a partial structure may be present in which two of the nitrogen atoms substituting the carbon atoms of the carbon material portion constituting the fused polycyclic aromatic moiety are linked to each other through two carbon atoms and are placed on the same side of the bond between the two carbon atoms. The partial structure including a nitrogen atom—a carbon atom—a carbon atom—a nitrogen atom may be present in the fused polycyclic aromatic moiety, and the two nitrogen atoms may be placed on the same side of the double bond between the two carbon atoms. The two nitrogen atoms may be placed at the cis-position to the double bond between the two carbon atoms. In the case where two nitrogen atoms are placed at the cis-position in the nitrogen-containing carbon material, the nitrogen-containing carbon material may include, for example, a 1,10-phenanthroline structure, a 4,7-phenanthroline structure, or a 2,9-phenanthroline structure. The content of the pyridinic nitrogen may thereby be increased in the nitrogen-containing carbon material and the nitrogen-containing carbon material may advantageously be used for various uses. In the case where two nitrogen atoms are placed at the cis-position in the nitrogen-containing carbon material, it is preferred that the nitrogen-containing carbon material include the 1,10-phenanthroline structure. The nitrogen-containing carbon material including the 1,10-phenanthroline structure is especially useful in the fields of a lithium sulfur battery, a carbon dioxide sensor, a redox reaction, a catalyst carrier and the like.

The fact that the nitrogen-containing carbon material includes the 1,10-phenanthroline structure may be confirmed by an XPS spectrum of the 3p-orbital of a metal element or an XPS analysis on the 1s orbital of nitrogen, utilizing the fact that the 1,10-phenanthroline structure tends to be coordinated by metal atoms. For example, the nitrogen-containing carbon material in which a metal atom is coordinated and another nitrogen-containing carbon material in which metal atom is coordinated are prepared, and an XPS spectrum of the 3p-orbital of the metal element or an XPS spectrum of the is orbital of nitrogen is measured for each of the above prepared two nitrogen-containing carbon material by using XPS. When these XPS spectra are compared to each other, variation of the binding energy may be observed, that is caused by the fact that the electronic state of the nitrogen atom at the 1,10-position differs depending on the presence or the absence of the coordinate with the metal atom. In the case where the metal atom is coordinated, when the variation of the binding energy in the XPS analysis may be observed, it may be confirmed that the nitrogen-containing carbon material includes the 1,10-phenanthroline structure. This may be confirmed by executing peak separation and fitting that take into consideration the variation of the peak position obtained when the pyridinic nitrogen is coordinated with the metal atom. The peak observed based on the coordinate with the metal atom may complexly be analyzed concurrently using, for example, IR spectroscopy, to distinguish from peaks derived from another classified nitrogen. The component necessary for the peak separation and the component unnecessary therefor may thereby be determined. The metal atom to be coordinated may be at least one selected from the group consisting of, for example, iron, cobalt, nickel, and copper. The metal atoms described herein may each be in the state such as a single atom, a metal cluster, a nanoparticle. A substance to be coordinated may also be an ion. In this case, an analysis may similarly be performed by the XPS analysis.

The nitrogen atoms in the fused polycyclic aromatic moiety of the nitrogen-containing carbon material may be pyridinic nitrogen atoms. The ratio of the pyridinic nitrogen atoms may be further increased by the fact that a partial structure like the above is included. The properties presented by the pyridinic nitrogen atoms that are the functions of, for example, adsorbing gas molecules and ionic chemical species, coordinating with a metal chemical species, a reduction activity for carbon dioxide and oxygen molecules, and the like may be imparted to the carbon material.

As to the nitrogen-containing carbon material, the carbon material includes a fused polycyclic aromatic moiety formed by condensation of three or more aromatic rings. The fused polycyclic aromatic moiety may include a partial structure in which two pyridinic nitrogen atoms are linked to each other through two carbon atoms and are placed on the same side of the bond between the two carbon atoms. The nitrogen-containing carbon material may include the partial structure in which two pyridinic nitrogen atoms are placed at a cis-position. This partial structure may be, for example, a 1,10-phenanthroline structure or a 2,9-phenanthroline structure, and may be preferably a 1,10-phenanthroline structure. The nitrogen-containing carbon material may include a partial structure, for example, like the one that is schematically represented by a chemical formula below.

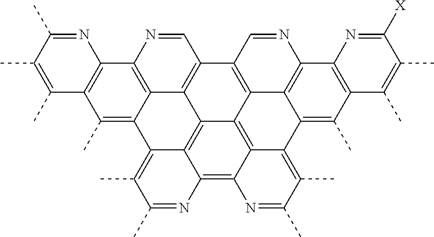

In the above chemical formula, X denotes a halogen atom, and a dotted line indicates a carbon-carbon bond and to be a partial structure. The position for a halogen atom to be present is not limited to the position indicated by the above chemical formula.

### Method for Producing Nitrogen-Containing Carbon Material

A method for producing the nitrogen-containing carbon material may include a first step of providing a nitrogen-containing aromatic compound that includes a phenanthroline structure and that includes a halogen atom as a substituent thereof, and a second step of carbonizing the nitrogen-containing aromatic compound by applying heat treatment thereto at 200° C. or higher and lower than 600° C. to obtain a carbonized heat-treated substance.

A nitrogen-containing carbon material may efficiently be produced to be the nitrogen-containing carbon material having a high content ratio of the pyridinic nitrogen atoms therein by using the nitrogen-containing aromatic compound that includes a phenanthroline structure and that includes a halogen atom as a substituent thereof, as the raw material of the nitrogen-containing carbon material. It may be considered that this is because, for example, the energy necessary for the carbonization reaction is reduced due to the halogen substitution in the nitrogen-containing aromatic compound to be the raw material and destruction of the raw material structure is thereby suppressed. The “nitrogen-containing aromatic compound including a phenanthroline structure” as used herein also encompasses materials each having, in a portion thereof, a partial structure similar to phenanthroline.

In the first step, a nitrogen-containing aromatic compound is provided. The nitrogen-containing aromatic compound includes a phenanthroline structure and includes a halogen atom as a substituent thereof. Examples of the halogen atom included, as the substituent, in the nitrogen-containing aromatic compound to be the raw material of the nitrogen-containing carbon material include a fluorine atom, a chlorine atom, a bromine atom, an iodine atom, and the like, and the nitrogen-containing aromatic compound includes at least one selected from the group consisting of the above. Examples of the halogen atom may preferably include at least one selected from the group consisting of a chlorine atom, a bromine atom, and an iodine atom. The number of substitutions of the halogen atoms in the nitrogen-containing aromatic compound may be, for example, 1 or greater and 8 or smaller. The number of substitutions of the halogen atoms may be, preferably 2 or greater and may be preferably 4 or smaller. The carbonization may thereby efficiently be advanced from the portion substituted by the halogen atom reducing the decomposition of the pyridinic nitrogen.

The nitrogen-containing aromatic compound includes at least pyridinic nitrogen atom. The number of contained pyridinic nitrogen atom in the nitrogen-containing aromatic compound may be 2 or greater and 8 or smaller in one molecule of the nitrogen-containing aromatic compound. The number of contained pyridinic nitrogen atom therein may be preferably 6 or smaller.

The nitrogen-containing aromatic compound may also include another substituent other than halogen atom. Examples of the other substituent include, for example, a hydroxy group, an alkoxy group, a cyano group, a nitro group, a carboxy group, a formyl group, an alkoxycarbonyl group, a carbamoyl group, a trifluoromethanesulfonyl group, a p-toluenesulfonyl group, and a diazonium group. In the case where the nitrogen-containing aromatic compound includes the other substituent other than halogen atom, the number of substitutions of the other substituents may be, for example, 4 or smaller, or 2 or smaller.

Examples of the nitrogen-containing aromatic compound to be the raw material of the nitrogen-containing carbon material include, for example, a compound that is represented by any one of formulae (1a) to (1d) below. The formula (1a) represents substituted 1,10-phenanthroline, the formula (1b) represents substituted 1,7-phenanthroline, the formula (1c) represents substituted 4,7-phenanthroline, and the formula (1d) represents substituted 2,9-phenanthroline.

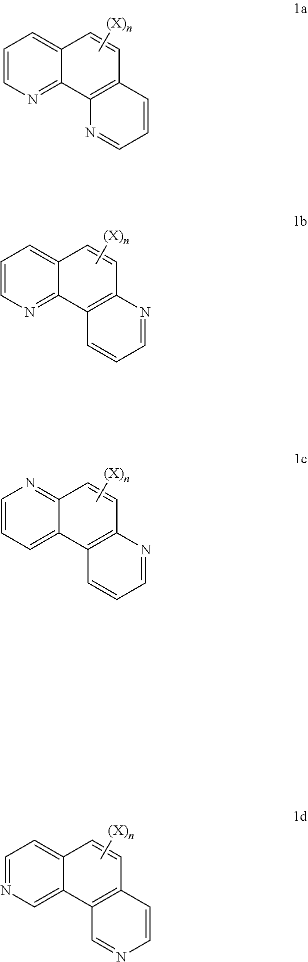

In the above formulae, X denotes a halogen atom. “n” is the number of substitutions of halogen atoms, and is indicated by a number that is 1 to 8. In the case where n is 2 or greater, the plural halogen atoms may be same as each other or may be different from each other, and may be substituted on the same ring or may be substituted on different rings. “n” may be a number that is preferably 1 to 6, or 1 to 4.

It is considered that each 1,7-phenanthroline, 4,7-phenanthroline, and 2,9-phenanthroline takes a form that takes structures similar to that of biphenyl without forming any six-membered ring among the raw materials due to the carbonization, and/or takes a form that takes honeycomb structures including benzene rings forming six-membered rings among the raw materials. The latter form is a graphene structure and may form the nitrogen-containing carbon material that includes a 1,10-phenanthroline structure. The nitrogen-containing carbon material may include a structure similar to biphenyl in a portion thereof.

It is preferred that the nitrogen-containing aromatic compound includes at least 1,10-phenanthroline that includes a halogen atom as a substituent. The nitrogen-containing carbon material including a 1,10-phenanthroline structure may be efficiently obtained by applying heat treatment to 1,10-phenanthroline that includes a halogen atom as a substituent. As to 1,10-phenanthroline, the pyridinic nitrogen tends to be changed to an amine-type nitrogen by thermal decomposition. Carbonization may easily be advanced from the portion that includes the halogen atoms maintaining the 1,10-phenanthroline structure at a high rate, by the fact that the nitrogen-containing aromatic compound includes halogen atoms as substituents. It is preferred that the nitrogen-containing aromatic compound include 1,10-phenanthroline that includes the halogen atom as the substituent. The nitrogen-containing carbon material including pyridinic nitrogen at a high content ratio may thereby be obtained.

Specific examples of a 1,10-phenanthroline derivative that includes a halogen atom as a substituent will be exemplified below as a specific example of the nitrogen-containing aromatic compound while the nitrogen-containing aromatic compound of the present disclosure is not limited to these. Examples of the nitrogen-containing aromatic compounds include, 2-chloro-1,10-phenanthroline, 2-bromo-1,10-phenanthroline, 2-iodo-1,10-phenanthroline, 3-chloro-1,10-phenanthroline, 3-bromo-1,10-phenanthroline, 3-iodo-1,10-phenanthroline, 4-chloro-1,10-phenanthroline, 4-bromo-1,10-phenanthroline, 4-iodo-1,10-phenanthroline, 5-chloro-1,10-phenanthroline, 5-bromo-1,10-phenanthroline, 5-iodo-1,10-phenanthroline, 4,7-dichloro-1,10-phenanthroline, 4,7-dibromo-1,10-phenanthroline, 4,7-diiodo-1,10-phenanthroline, 2,9-dichloro-1,10-phenanthroline, 2,9-dibromo-1,10-phenanthroline, 2,9-diiodo-1,10-phenanthroline, 3,8-dichloro-1,10-phenanthroline, 3,8-dibromo-1,10-phenanthroline, 3,8-diiodo-1,10-phenanthroline, 5,6-dichloro-1,10-phenanthroline, 5,6-1,10-dibromo-phenanthroline, 5,6-diiodo-1,10-phenanthroline, 3,5,6,8-tetrachloro-1,10-phenanthroline, 3,5,6,8-tetrabromo-1,10-phenanthroline, and 3,5,6,8-tetraiodo-1,10-phenanthroline.

Specific examples of 1,7-phenanthroline, 2,9-phenanthroline, and 4,7-phenanthroline that each includes a halogen atom as a substituent will be exemplified below as the nitrogen-containing aromatic compound while the nitrogen-containing aromatic compound of the present disclosure is not limited to these. The nitrogen-containing aromatic compounds include 3-bromo-1,7-phenanthroline, 5-bromo-2,9-phenanthroline, and 2-bromo-4,7-phenanthroline.

The nitrogen-containing aromatic compound may be purchased to be provided or may be produced using a known method to be provided. The nitrogen-containing aromatic compound substituted by a halogen atom may be produced by, for example, a halogen substitution reaction for an available halogenated nitrogen-containing aromatic compound, a halogenation reaction for a nitrogen-containing aromatic compound, and the like.

In the second step, the nitrogen-containing aromatic compound is heat treated at 200° C. or higher and lower than 600° C. to be carbonized to obtain a nitrogen-containing carbon material as a carbonized heat-treated substance. The nitrogen-containing carbon material may be produced by carbonizing the nitrogen-containing aromatic compound by applying thereto heat treatment. The carbonization means production of a carbon material whose content amount of carbon is 50 at % or higher and for which the G-band is observed by Raman spectroscopy, by applying heat treatment to the nitrogen-containing aromatic compound. The content amount of carbon is determined from the composition ratios determined by the elemental analysis. It is considered that the heat treatment is applied to the nitrogen-containing aromatic compound and thereby, for example, halogen atoms, hydrogen atoms, and the like are removed from the nitrogen-containing aromatic compound and a carbon-carbon bond is newly formed, and the carbonization is thereby performed.

The heat treatment temperature for heat-treating the nitrogen-containing aromatic compound may be a temperature, for example, in a range of 200° C. or higher and lower than 600° C. The heat treatment temperature may be, preferably 230° C. or higher, 250° C. or higher, 280° C. or higher, or 300° C. or higher, and may also be preferably 550° C. or lower, 500° C. or lower, 450° C. or lower, or 400° C. or lower. The controllability of the structure of the phenanthroline structure may further be enhanced by the fact that the heat treatment temperature is in the above ranges. The content ratio of the pyridinic nitrogen atoms thereby tends to be improved.

The heat treatment for the nitrogen-containing aromatic compound may be performed by, for example, increasing the temperature from the room temperature to a predetermined heat treatment temperature and maintaining the predetermined heat treatment temperature for a predetermined heat treatment time period. The temperature increase rate may be, for example, 1° C./min or higher and 30° C./min or lower, and preferably 5° C./min or higher and 15° C./min or lower. The heat treatment time period may be, for example, 30 minutes or longer and 24 hours or shorter, and may be preferably 30 minutes or longer and 6 hours or shorter.

The heat treatment for the nitrogen-containing aromatic compound may be performed under a sealed tube condition. For example, sublimation of the raw material compound may be suppressed by executing the heat treatment under the sealed tube condition. The heat treatment for the nitrogen-containing aromatic compound may be performed under a reduced pressure. The side reactions caused by, for example, the gases generated associated with the carbonization may be suppressed by executing the heat treatment under a reduced pressure. The reduced pressure condition for the heat treatment may be for example 50 Pa or lower, preferably 30 Pa or lower, more preferably 10 Pa or lower, and especially preferably 1 Pa or lower. The reduced pressure condition may be, for example, 0.1 Pa or higher.

In the heat treatment for the nitrogen-containing aromatic compound, the nitrogen-containing aromatic compound may be brought into contact with powder or a substrate to perform the heat treatment at a temperature in a range of 200° C. or higher and lower than 600° C. In the heat treatment for the nitrogen-containing aromatic compound, the nitrogen-containing aromatic compound may also be supported on powder or a substrate to perform the heat treatment at a temperature in a range of 200° C. or higher and lower than 600° C. The material of the powder or the substrate may be, for example, copper, nickel, cobalt, platinum, gold, or the like that presents a catalyst action. The pyridinic nitrogen atoms thereby bond with the powder or the substrate made from the above, and the decomposition of the pyridinic nitrogen atoms due to the heat treatment may thereby be reduced. The particle diameter of the metal powder that presents a catalyst action may be, for example, 1 nm or larger and 300 nm or smaller and may be 50 nm or larger and 100 nm or smaller. The material of the powder or the substrate may also be graphite, carbon black, carbon nanotubes, activated carbon, silicon oxide, aluminum oxide, glass, or the like.

A method for producing the nitrogen-containing carbon material may further include a crushing step of crushing a heat-treated substance after the heat treatment step, a purification step of purifying the heat-treated substance, and the like.

The purification step may include a first purification step of removing the impurities in the obtained heat-treated substance, and a second purification step of removing the residual metals that are not completely removed at the first purification step. The residual metals may be removed using, for example, an acid, a chelating agent, or a metal scavenger.

### APPLICATION EXAMPLES

A fuel battery may include the nitrogen-containing carbon material according to the present disclosure. An electrode of a lithium sulfur battery may include the nitrogen-containing carbon material according to the present disclosure. A carbon dioxide sensor may include the nitrogen-containing carbon material according to the present disclosure. A catalyst carrier may include the nitrogen-containing carbon material according to the present disclosure. An adsorbing material for carbon dioxide may include the nitrogen-containing carbon material according to the present disclosure.

### Examples

The present disclosure will be described below in detail with reference to Examples while the present disclosure is not limited to these Examples.

### Synthesis Example 1 of Nitrogen-Containing Aromatic Compound

### Synthesis of 2,9-Diiodo-1,10-Phenanthroline (29-DIP)

2,9-Dichloro-1,10-phenanthroline (498 mg, manufactured by Tokyo Chemical Industry Co., Ltd.) and sodium iodide (900 mg, manufactured by FUJIFILM Wako Pure Chemical Corp.) were put in a Schlenk tube. The atmosphere in the Schlenk tube was replaced with nitrogen and a hydrogen iodide aqueous solution of 55% by mass (12 ml, manufactured by FUJIFILM Wako Pure Chemical Corp.) was thereafter added to be caused to react with each other at 80° C. for 24 hours. After the reaction came to an end, a reaction mixture was suction-filtrated and a solid crude product was washed using distilled water (150 ml) and 2-propanol (30 ml, manufactured by Kanto Chemical Co., Inc.). The crude product was dissolved in dimethyl sulfoxide (35 ml, manufactured by Kanto Chemical Co., Inc.) and was membrane-filtrated to be thereafter mixed with a potassium iodide aqueous solution that was prepared by dissolving potassium iodide (535 mg, manufactured by Sigma-Aldrich Co., LLC) in distilled water (20 ml), to be stirred at the room temperature for 30 minutes. A precipitated crystalline white solid was collected by filtration and the collected white solid was thereafter washed using distilled water (50 ml) and 2-propanol (30 ml, manufactured by Kanto Chemical Co., Inc.), and was dried at a reduced pressure to obtain purified 2,9-diiodo-1,10-phenanthroline (710 mg).

### Synthesis Example 2 of Nitrogen-Containing Aromatic Compound

### Synthesis of 4,7-Diiodo-1,10-Phenanthroline (47-DIP)

4,7-Dichloro-1,10-phenanthroline (500 mg, manufactured by Tokyo Chemical Industry Co., Ltd.) and sodium iodide (900 mg, manufactured by FUJIFILM Wako Pure Chemical Corp.) were put in a Schlenk tube. The atmosphere in the Schlenk tube was replaced with nitrogen and a hydrogen iodide aqueous solution of 55% by mass (12 ml, manufactured by FUJIFILM Wako Pure Chemical Corp.) was thereafter added to be caused to react with each other at 80° C. for 12 hours. After the reaction came to an end, distilled water (30 ml) was put into a reaction mixture to be suction-filtrated, and a solid crude product was washed using distilled water (150 ml) and 2-propanol (3 ml, manufactured by Kanto Chemical Co., Inc.). After the washing, the crude product was dissolved in dimethyl sulfoxide (80 ml, manufactured by Kanto Chemical Co., Inc.) and was membrane-filtrated to be mixed with a potassium iodide aqueous solution that was prepared by dissolving potassium iodide (900 mg, manufactured by Sigma-Aldrich Co., LLC) and potassium hydroxide (350 mg, manufactured by Kanto Chemical Co., Inc.) in distilled water (50 ml), to be stirred at the room temperature for 30 minutes. A precipitated needle-like white solid was collected by filtration and the needle-like white solid was washed using distilled water (350 ml) and 2-propanol (30 ml, manufactured by Kanto Chemical Co., Inc.) and was dried at a reduced pressure to obtain purified 4,7-diiodo-1,10-phenanthroline (540 mg).

### Synthesis Example 3 of Nitrogen-Containing Aromatic Compound

### Synthesis of 2,9-Dibromo-1,10-Phenanthroline (29-DBP)

2,9-Dichloro-1,10-phenanthroline (750 mg, manufactured by Tokyo Chemical Industry Co., Ltd.) was put in a Schlenk tube. The atmosphere in the Schlenk tube was replaced with nitrogen and phosphorus tribromide (5 ml, manufactured by Sigma-Aldrich Co., LLC) was thereafter added to be caused to react with each other at 170° C. for 6 hours. After the reaction, distilled water was added thereto cooling the Schlenk tube with ice to neutralize the reaction mixture using sodium carbonate (manufactured by FUJIFILM Wako Pure Chemical Corp.). The reaction mixture was suction-filtrated and the obtained crude product was thereafter washed using distilled water (500 ml) and methanol (3 ml, manufactured by Kanto Chemical Co., Inc.) that was cooled by ice. The crude product after the washing was dried at a reduced pressure and was dissolved being heated in methanol (190 ml, manufactured by Kanto Chemical Co., Inc.) and was left untouched at 4° C. to complete recrystallization thereof to thereby obtain purified 2,9-dibromo-1,10-phenanthroline (625 mg).

### Synthesis Example 4 of Nitrogen-Containing Aromatic Compound

### Synthesis of 5,6-Dibromo-1,10-Phenanthroline (56-DBP)

1,10-Phenanthroline (1.1 g, manufactured by Tokyo Chemical Industry Co., Ltd.), 30%-oleum (9 ml, manufactured by FUJIFILM Wako Pure Chemical Corp.), and bromine (0.3 ml, manufactured by Tokyo Chemical Industry Co., Ltd.) were mixed with each other being cooled by ice, to be caused to react with each other in a pressure-resistant container made from SUS at 120° C. for 22 hours. After the reaction came to an end, distilled water (380 ml) was added thereto being cooled by ice to prepare pH to be 3. A reaction mixture was suction-filtrated and the obtained crude product was thereafter extracted using dichloromethane (250 ml, manufactured by Kanto Chemical Co., Inc.) to be shunt-washed with distilled water. The collected organic layer was dried using magnesium sulfate (manufactured by FUJIFILM Wako Pure Chemical Corp.) to distill away the solvent. The residue was dissolved being heated in ethanol (20 ml, manufactured by Kanto Chemical Co., Inc.) to be thereafter left untouched at 4° C. to complete recrystallization thereof to thereby obtain purified 5,6-dibromo-1,10-phenanthroline (560 mg).

### Providing of Nitrogen-Containing Aromatic Compound

A commercially available nitrogen-containing aromatic compound (manufactured by Tokyo Chemical Industry Co., Ltd.) was used as each of the nitrogen-containing aromatic compounds other than those of the above Synthesis Examples 1 to 4 of the nitrogen-containing aromatic compound, and Example 8. A commercially available nitrogen-containing aromatic compound (manufactured by Sigma-Aldrich Co., LLC) was used as the nitrogen-containing aromatic compound of Example 8. In only Example 10 described later, commercially available 4,7-dibromo-1,10-phenanthroline was purified to obtain the nitrogen-containing aromatic compound.

### Example 1

Sixty mg of 2,9-dichloro-1,10-phenanthroline (29-DCP; manufactured by Tokyo Chemical Industry Co., Ltd.) was put in a glass tube as the nitrogen-containing aromatic compound, to be dried at a reduced pressure at 120° C. for 1 hour. Maintaining the reduced pressure, the glass tube was sealed using a gas burner such that the length of the glass tube was about 7 cm to make an ampoule tube. The ampoule tube was put in a tube electric furnace, and the temperature thereof was increased from the room temperature (about 25° C.) to 400° C. at a temperature increase rate of 10° C./min to apply thereto heat treatment for 1 hour to obtain a carbonized substance. After the heat treatment, the carbonized substance was collected and was finely ground using an agate mortar, and the pressure was reduced at 300° C. for 1 hour to thereby remove the raw material and a low-molecular weight component to obtain a specimen that was the nitrogen-containing carbon material of Example 1.

### Examples 2 to 14

Each specimen to be the nitrogen-containing carbon material was obtained in the same manner as that of Example 1 except the fact that the nitrogen-containing aromatic compounds shown in Table 1 were used instead of 2,9-dichloro-1,10-phenanthroline and that the heat treatment temperature was changed as shown in Table 1.

The abbreviations in Table 1 mean as described below.

1,10-phen: 1,10-phenanthroline

29-DCP: 2,9-dichloro-1,10-phenanthroline

29-DBP: 2,9-dibromo-1,10-phenanthroline

29-DIP: 2,9-diiodo-1,10-phenanthroline

47-DCP: 4,7-dichloro-1,10-phenanthroline

47-DBP: 4,7-dibromo-1,10-phenanthroline

47-DIP: 4,7-diiodo-1,10-phenanthroline

38-DBP: 3,8-dibromo-1,10-phenanthroline

3568-TBP: 3,5,6,8-tetrabromo-1,10-phenanthroline

56-DBP: 5,6-dibromo-1,10-phenanthroline

5-CP: 5-chloro-1,10-phenanthroline

### Comparative Example 1

A specimen of Comparative Example 1 was obtained in the same manner as that of Example 1 except the fact that 1,10-phenanthroline (1,10-Phen; manufactured by Tokyo Chemical Industry Co., Ltd.) was used instead of 2,9-dichloro-1,10-phenanthroline.

### Evaluation

### Composition and Content of Nitrogen

For each of the specimens obtained in Examples 1 to 14, the composition and the content of nitrogen were evaluated as below. The ratio of each of carbon, nitrogen, and hydrogen in each of the specimens was analyzed by a combustion method of an elemental analysis. Carbon was obtained by analyzing the amount of carbon dioxide, hydrogen was obtained by analyzing the amount of water, and nitrogen was obtained by analyzing the amount of a nitrogen gas. The composition ratios were determined by dividing the value in % by mass (wt %) of each of the elements obtained by the elemental analysis, by the atomic weight of the element. Because phenanthroline includes 12 carbon atoms, the composition ratios were determined assuming that 12 carbon atoms were included. The atomic weight percent (at %) of nitrogen was determined from the determined composition ratios. Table 1 shows the result for the nitrogen content ratio (at %).

### Content of Pyridinic Nitrogen Atom

For each of the specimens obtained in Examples 1 to 14, the content of the pyridinic nitrogen atoms in the nitrogen atoms was evaluated in the following manner.

An XPS spectrum of the is orbital of nitrogen was measured using XPS. XPS was performed in a vacuum chamber at a degree of vacuum of 2×10⁻⁵ Pa. AXIS-ULTRA DLD (Shimadzu Corp.) was used for the XPS spectrum measurement. The XPS measurement was performed at the room temperature using a specimen placed on a carbon table as the specimen for the measurement. Such measurement conditions were employed as a light source of MgKα-line (dual anode), an emission current of 10 mA, an anode voltage of 10 kV, path energy of 40 eV, a measurement range of 385 eV or higher and 409 eV or lower converted into the binding energy, and the number of integration sessions of 10. For each specimen charged up when the XPS spectrum was measured, a neutralizing gun was used under such neutralization conditions as a filament current of 1.75 A, a charge balance of 3.0 V, and a filament bias of 1.0 V.

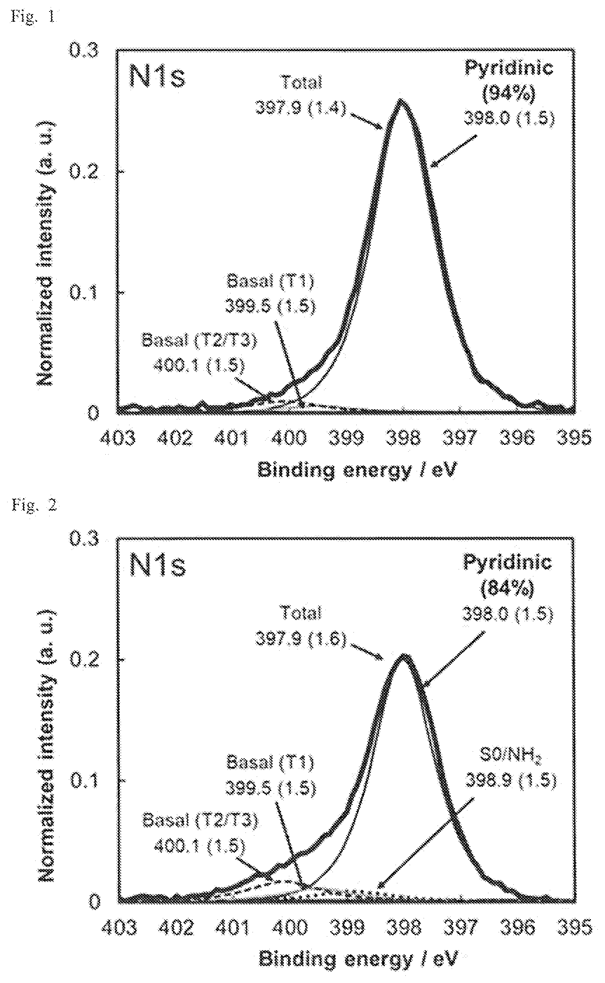

The obtained XPS spectrum of the is orbital of nitrogen was analyzed as follows. The measured kinetic energy of the photoelectron was first converted into binding energy. Shirley background was assumed as the background and was removed, and peak separation and fitting were performed. The peak separation was performed assuming that the binding energy of the pyridinic nitrogen had the peak top thereof at 398.0 eV, the binding energy of the basal nitrogen (T1) had the peak top thereof at 399.5 eV, and the binding energy of basal nitrogen (T2, T3) had the peak top thereof at 400.1 eV. Using these pieces of binding energy, a Voigt function was set and an asymmetry function was added thereto to perform the fitting. The full width at half maximum of each peak was set to be 1.5 eV. The content ratio of the pyridinic nitrogen was determined from the area ratio of the overall area of the XPS spectrum of the is orbital of nitrogen to the area of the XPS spectrum of the is orbital of nitrogen derived from the pyridinic nitrogen. FIG. 1 and FIG. 2 depict an example of the peak separation. Table 1 shows the result of the content ratio of the pyridinic nitrogen atoms estimated based on the XPS for each of Examples 1 to 14. For Examples 3 to 6 and Examples 8 to 14, amine-type nitrogen (S0, NH₂) was additionally included in the peak separation and the fitting. It was assumed that the binding energy of the amine-type nitrogen had the peak top thereof at 398.9 eV. The full width at half maximum of each of the peaks used in the peak separation was set to be 1.5 eV. For Examples 4 to 11, Example 13, and Example 14, the quaternary nitrogen (Q-N) was additionally included in the peak separation and the fitting. It was assumed that the binding energy of the quaternary nitrogen had the peak top thereof at 401.2 eV. The full width at half maximum of each of the peaks was set to be 1.5 eV.

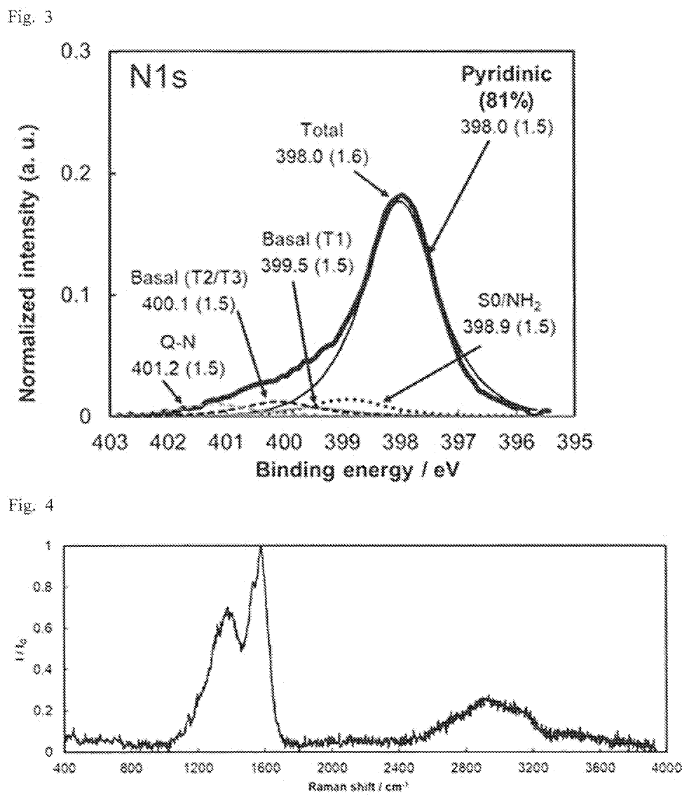

FIG. 1 is the measurement result of an XPS spectrum of the is orbital of nitrogen in the nitrogen-containing carbon material of Example 2. It was confirmed from the result of the peak separation that the content ratio of the pyridinic nitrogen atoms was 94%. FIG. 2 is the measurement result of XPS spectra of the is orbital of nitrogen in the nitrogen-containing carbon material of Example 3. It was confirmed from the result of the peak separation that the content ratio of the pyridinic nitrogen atoms was 84%. FIG. 3 is the measurement result of XPS spectra of the is orbital of nitrogen in the nitrogen-containing carbon material of Example 5. It was confirmed from the result of the peak separation that the content ratio of the pyridinic nitrogen atoms was 81%.

TABLE 1

| | | | | | Content of |
|---|---|---|---|---|---|
| | | Heat-treating | | Content of | pyridinic |
| | Raw | temperature | | nitrogen | nitrogen |
| | material | (° C.) | Composition | (at %) | atom (%) |
| Example 1 | 29-DCP | 400 | C₁₂H_{5.83}N_{2.24}Cl_{1.36} | 10.4 | 69 |
| Example 2 | 47-DCP | 300 | C₁₂H_{5.47}N_{2.13}Cl_{1.50} | 10.1 | 94 |
| Example 3 | 47-DCP | 400 | C₁₂H_{5.23}N_{2.02}Cl_{0.69} | 10.1 | 84 |
| Example 4 | 29-DIP | 300 | C₁₂H_{5.85}N_{1.91}I_{0.69} | 9.3 | 64 |
| Example 5 | 47-DIP | 300 | C₁₂H_{8.46}N_{2.03}I_{0.51} | 8.8 | 81 |
| Example 6 | 47-DIP | 400 | C₁₂H_{7.59}N_{2.00}I_{0.55} | 9.0 | 64 |
| Example 7 | 38-DBP | 400 | C₁₂H_{4.91}N_{1.95}Br_{0.31} | 10.2 | 76 |
| Example 8 | 3568-TBP | 400 | C₁₂H_{2.96}N_{2.01}Br_{1.93} | 10.7 | 68 |
| Example 9 | 29-DBP | 400 | C₁₂H_{5.69}N_{2.00}Br_{0.38} | 10.0 | 67 |
| Example 10 | 47-DBP | 400 | C₁₂H_{5.98}N_{1.99}Br_{0.47} | 9.7 | 75 |
| Example 11 | 56-DBP | 400 | C₁₂H_{5.57}N_{2.00}Br_{0.36} | 10.0 | 63 |
| Example 12 | 47-DCP | 500 | C₁₂H_{3.85}N_{1.90}Cl_{0.99} | 10.1 | 61 |
| Example 13 | 5-CP | 500 | C₁₂H_{6.22}N_{1.62}Cl_{0.66} | 7.9 | 72 |
| Example 14 | 38-DBP | 500 | C₁₂H_{4.1}N_{2.0}Br_{0.6} | 10.7 | 68 |

As shown in Table 1, it was confirmed that the nitrogen-containing carbon material having a high content ratio of nitrogen was able to be obtained by using, as the raw material, the nitrogen-containing aromatic compound that included a phenanthroline structure and that included a halogen atom as a substituent thereof. It was also confirmed that the nitrogen-containing carbon material having a high content ratio of the pyridinic nitrogen atoms was able to be obtained. Only the production of an oil-like substance was recognized for the specimen of Comparative Example 1 and no nitrogen-containing carbon material was obtained, and no composition analysis was therefore performed. As a result of ¹H-nuclear magnetic resonance (NMR) measurement, it turned out that a compound including an amine-type nitrogen was present in the specimen of Comparative Example 1. It may be considered that this indicates that no carbonization was advanced in Comparative Example 1 and the raw material was thermally decomposed. For the ¹H-NMR measurement, a nuclear magnetic resonance device (JNM-ECA500; manufactured by JEOL Ltd.) was used. The measurement conditions were set as the resonance frequency of 500 MHz and the pulse width of 7.11 μsec.

### Raman Spectroscopy Measurement

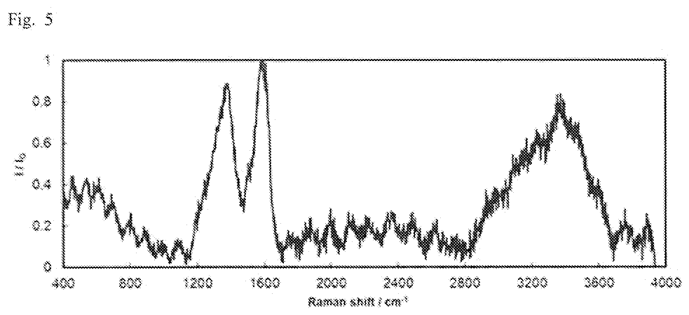

A Raman spectroscopy spectrum was measured for each specimen obtained in Examples 1 and 3 using a micro-laser Raman spectrophotometer (NRS-4500; manufactured by JASCO Corp.). The wavelength of the excitation light source was 532 nm. The intensity of the laser was 0.3 mW. The magnification of the objective lens was set to be 100×. The exposure time was 10 sec or 20 sec, and the number of integration sessions in the measurement of each spectrum was 10. FIGS. 4 and 5 depict the result of the above. The spectra were normalized using the peak presenting the maximal intensity.

From the result of the Raman spectroscopy, peaks were recognized in a range of 1,300 cm⁻¹ or higher and 1,400 cm⁻¹ or lower and a range of 1,570 cm⁻¹ or higher and 1,600 cm⁻¹ or lower. These were the peaks reflecting the D-band and the G-band, and it was presumed that the specimens obtained in Examples 1 and 3 were carbonized.

### Example 15

3,5,6,8-Tetrabromo-1,10-phenanthroline that was a commercially available product (manufactured by Sigma-Aldrich Co., LLC) was used as a raw material of the nitrogen-containing carbonized substance. Twenty mg of 3,5,6,8-tetrabromo-1,10-phenanthroline and copper particle powder whose particle diameter was 60 nm to 80 nm (400 mg, manufactured by Sigma-Aldrich Co., LLC) were put in a glass tube and were dried at a reduced pressure at 180° C. for 1 hour. While maintaining the reduced pressure, the glass tube was sealed using a gas burner such that the length of the glass tube was about 7 cm to make an ampoule tube. The ampoule tube was put in a tube electric furnace, and the temperature thereof was increased from the room temperature (about 25° C.) to 450° C. at a temperature increase rate of 10° C./min to apply thereto heat treatment to obtain a carbonized substance. The ampoule tube was opened and the carbonized substance was collected. The collected carbonized substance was heated under the reduced pressure for 1 hour at 380° C. to thereby remove the raw material and a low-molecular weight component to obtain a specimen that was the nitrogen-containing carbon material of Example 15. For the obtained specimen, an XPS analysis and the peak separation were performed in the same manner as that of Examples 1 to 14. It was able to be confirmed by the peak separation and the fitting that the content of the pyridinic nitrogen atoms was higher than at least 59%.

It is to be understood that although the present invention has been described with regard to preferred embodiments thereof, various other embodiments and variants may occur to those skilled in the art, which are within the scope and spirit of the invention and such other embodiments and variants are intended to be covered by the following claims.

Although the present disclosure has been described with reference to several exemplary embodiments, it is to be understood that the words that have been used are words of description and illustration, rather than words of limitation. Changes may be made within the purview of the appended claims, as presently stated and as amended, without departing from the scope and spirit of the disclosure in its aspects. Although the disclosure has been described with reference to particular examples, means, and embodiments, the disclosure may be not intended to be limited to the particulars disclosed; rather the disclosure extends to all functionally equivalent structures, methods, and uses such as are within the scope of the appended claims.

One or more examples or embodiments of the disclosure may be referred to herein, individually and/or collectively, by the term “disclosure” merely for convenience and without intending to voluntarily limit the scope of this application to any particular disclosure or inventive concept. Moreover, although specific examples and embodiments have been illustrated and described herein, it should be appreciated that any subsequent arrangement designed to achieve the same or similar purpose may be substituted for the specific examples or embodiments shown. This disclosure may be intended to cover any and all subsequent adaptations or variations of various examples and embodiments. Combinations of the above examples and embodiments, and other examples and embodiments not specifically described herein, will be apparent to those of skill in the art upon reviewing the description.

In addition, in the foregoing Detailed Description, various features may be grouped together or described in a single embodiment for the purpose of streamlining the disclosure. This disclosure may not be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above-disclosed subject matter shall be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure may be determined by the broadest permissible interpretation of the following claims and their equivalents, and shall not be restricted or limited by the foregoing

### DETAILED DESCRIPTION

All publications, patent applications, and technical standards mentioned in this specification are herein incorporated by reference to the same extent as if each individual publication, patent application, or technical standard was specifically and individually indicated to be incorporated by reference.

What is claimed is:

1. A nitrogen-containing carbon material comprising: carbon atoms; nitrogen atoms; and halogen atoms; wherein
a ratio of a number of moles of pyridinic nitrogen atoms to a total number of moles of the nitrogen atoms is higher than 59%, wherein
a total content ratio of the nitrogen atoms with respect to the nitrogen-containing carbon material is 7 at % or higher, wherein
the nitrogen-containing carbon material comprises a fused polycyclic aromatic moiety formed by condensation of three or more aromatic rings, and wherein
the fused polycyclic aromatic moiety comprises a partial structure for two of the pyridinic nitrogen atoms to be linked to each other through two of the carbon atoms.

2. The nitrogen-containing carbon material according to claim 1, wherein
a content ratio of the halogen atoms with respect to the nitrogen-containing carbon material is 0.01 at % or higher and 30 at % or lower.

3. The nitrogen-containing carbon material according to claim 1, wherein
the halogen atoms comprise at least one selected from the group consisting of chlorine atoms, bromine atoms, and iodine atoms.

4. The nitrogen-containing carbon material according to claim 2, wherein
the halogen atoms comprise at least one selected from the group consisting of chlorine atoms, bromine atoms, and iodine atoms.

5. The nitrogen-containing carbon material according to claim 1, wherein
the two pyridinic nitrogen atoms are placed on the same side of a bond between the two carbon atoms.

6. The nitrogen-containing carbon material according to claim 2, wherein
the two pyridinic nitrogen atoms are placed on the same side of a bond between the two carbon atoms.

7. The nitrogen-containing carbon material according to claim 3, wherein
the two pyridinic nitrogen atoms are placed on the same side of a bond between the two carbon atoms.

8. A method for producing a nitrogen-containing carbon material, the method comprising:
providing a nitrogen-containing aromatic compound that comprises a phenanthroline structure and that comprises a halogen atom as a substituent thereof; and
carbonizing the nitrogen-containing aromatic compound by applying a heat treatment thereto at 200° C. or higher and lower than 600° C.

9. The method according to claim 8, wherein
the halogen atom comprises at least one selected from the group consisting of a chlorine atom, a bromine atom, and an iodine atom.

10. The method according to claim 8, wherein
the nitrogen-containing aromatic compound comprises 1,10-phenanthroline comprising a halogen atom as a substituent.

11. The method according to claim 9, wherein
the nitrogen-containing aromatic compound comprises 1,10-phenanthroline comprising a halogen atom as a substituent.

12. The method according to claim 8, wherein
the heat treatment is performed under a reduced pressure.

13. The method according to claim 9, wherein
the heat treatment is performed under a reduced pressure.

14. The method according to claim 10, wherein
the heat treatment is performed under a reduced pressure.
